SEQUENCE LISTING (21 sequences)

SEQ ID NO: 1 - protein, 4 aa, unknown
  Poultry
PVLK

SEQ ID NO: 2 - protein, 4 aa, unknown
  Poultry
PLPR

SEQ ID NO: 3 - protein, 5 aa, unknown
  Poultry
EAEFD

SEQ ID NO: 4 - protein, 5 aa, unknown
  Poultry
PERNE

SEQ ID NO: 5 - protein, 5 aa, unknown
  Poultry
PQPER

SEQ ID NO: 6 - protein, 8 aa, unknown
  Poultry
EFDEKPAD

SEQ ID NO: 7 - protein, 7 aa, unknown
  Poultry
EDAPVLH

SEQ ID NO: 8 - protein, 7 aa, unknown
  Poultry
EFDARPT

SEQ ID NO: 9 - protein, 7 aa, unknown
  Poultry
EETPSHD

SEQ ID NO: 10 - protein, 9 aa, unknown
  Poultry
EEVEEEEVE

SEQ ID NO: 11 - protein, 8 aa, unknown
  Poultry
EFDEKAPD

SEQ ID NO: 12 - protein, 7 aa, unknown
  Poultry
ENAPDQK

SEQ ID NO: 13 - protein, 5 aa, unknown
  Poultry
EDMAP

SEQ ID NO: 14 - protein, 5 aa, unknown
  Poultry
PINDN

SEQ ID NO: 15 - protein, 4 aa, unknown
  Poultry
PTDL

SEQ ID NO: 16 - protein, 4 aa, unknown
  Poultry
PVAE

SEQ ID NO: 17 - protein, 5 aa, unknown
  Poultry
RNGPR

SEQ ID NO: 18 - protein, 9 aa, unknown
  Poultry
RAEDTATYY

SEQ ID NO: 19 - protein, 7 aa, unknown
  Poultry
EGDSAAI

SEQ ID NO: 20 - protein, 8 aa, unknown
  Poultry
EPEGDVHQ

SEQ ID NO: 21 - protein, 8 aa, unknown
  Poultry
EDEVLATP

(19) United States
(12) Patent Application Publication
(10) Pub. No.: US 2026/0055137 A1
(43) Pub. Date: Feb. 26, 2026

(54) NOVEL ISOLATED PEPTIDES, PROTEIN HYDROLYSATE COMPRISING THE SAID ISOLATED PEPTIDES AND USE THEREOF

(71) Applicant: BETAGRO PUBLIC COMPANY LIMITED, Bangkok (TH)

(72) Inventors: Tewa UPATHANPREECHA, Pathum Thani (TH); Sintawee SULAIMAN, Bangkok (TH); Tiandan WU, Muenchen (DE); Patthana TASTUB, Chai Nat (TH); Verena Karolin MITTERMEIER-KLESSINGER, Garching (DE); Corinna DAWID, Freising (DE); Thomas HOFMANN, Muenchen (DE); Andreas DUNKEL, Freising (DE)

(73) Assignee: BETAGRO PUBLIC COMPANY LIMITED, Bangkok (TH)

(21) Appl. No.: 18/864,910

(22) PCT Filed: May 13, 2022
(86) PCT No.: PCT/IB2022/054473

§ 371 Date: Nov. 12, 2024

(51) Int. Cl. C07K 5/117; A23L 27/00; A23L 27/10; C07K 7/06; C12N 9/64

(52) U.S. Cl. CPC C07K 5/1024; A23L 27/10; A23L 27/88; C07K 7/06; C12N 9/6424; C12Y 304/21062

(57) ABSTRACT

The invention relates to an isolated peptide having an amino acid sequence selected from the group consisting of: Pro-Val-Leu-Lys (SEQ ID NO: 1); Pro-Leu-Pro-Arg (SEQ ID NO: 2); Glu-Ala-Glu-Phe-Asp (SEQ ID NO: 3); Pro-Glu-Arg-Asn-Glu (SEQ ID NO: 4); Pro-Gln-Pro-Glu-Arg (SEQ ID NO: 5); Glu-Phe-Asp-Glu-Lys-Pro-Ala-Asp (SEQ ID NO: 6); Glu-Asp-Ala-Pro-Val-Leu-His (SEQ ID NO: 7); Glu-Phe-Asp-Ala-Arg-Pro-Thr (SEQ ID NO: 8); Glu-Glu-Thr-Pro-Ser-His-Asp (SEQ ID NO: 9); Glu-Glu-Val-Glu-Glu-Glu-Glu-Val-Glu (SEQ ID NO: 10); Glu-Phe-Asp-Glu-Lys-Ala-Pro-Asp (SEQ ID NO: 11); Glu-Asn-Ala-Pro-Asp-Gln-Lys (SEQ ID NO: 12); Glu-Asp-Met-Ala-Pro (SEQ ID NO: 13); Pro-Ile-Asn-Asp-Asn (SEQ ID NO: 14); Pro-Thr-Asp-Leu (SEQ ID NO: 15); Pro-Val-Ala-Glu (SEQ ID NO: 16); Arg-Asn-Gly-Pro-Arg (SEQ ID NO: 17); Arg-Ala-Glu-Asp-Thr-Ala-Thr-Tyr-Tyr (SEQ ID NO: 18); Glu-Gly-Asp-Ser-Ala-Ala-Ile (SEQ ID NO: 19); Glu-Pro-Glu-Gly-Asp-Val-His-Gln (SEQ ID NO: 20); and Glu-Asp-Glu-Val-Leu-Ala-Thr-Pro (SEQ ID NO: 21), a protein hydrolysate comprising the isolated peptides, and use thereof as a taste-modulating agent in food and pharmaceutical preparations.

### TECHNICAL FIELD

The present invention relates to novel isolated peptides, a protein hydrolysate comprising the said isolated peptides for use as a taste-modulating agent in food and pharmaceutical preparations.

### BACKGROUND OF THE INVENTION

Livestock and poultry blood is a side product obtained from the industrial slaughter process. A large volume of livestock and poultry blood, e.g., chicken blood is produced every year and wasted in many countries around the world, despite its valuable proteins and the bioavailability of the nutrients. Therefore, the utilization of such blood is advantageous in terms of both economy and environment. Until now, the utilization of the livestock and poultry blood is limited, regardless of the cost efficiency or the feeding goal since the use of whole blood and proteins is restricted in the food industry due to unpleasant flavor or color. To address these problems, some studies have been conducted to recycle byproducts and waste materials obtained from poultry processing and industry. For example, chicken blood is used to make blood sausage, blood tofu, and blood pudding. Blood plasma could be used as a protein supplement, which is an excellent source of trace minerals, or as a replacement of egg white in the baking industry due to its great foaming capacity (Jayathilakan et al., J Food Sci Technol, 2012, 49 (3), 378-293).

To increase the use of chicken blood, the generation of taste-active and taste-modulating peptides is proposed. However, the existing knowledge of chicken blood composition, e.g., which proteins are presented or the accurate concentrations of several constituents, is limited. Besides, there is no study available related to the taste of chicken blood, although many studies have been performed to identify the taste-active and taste-modulating peptides in all kinds of food. Due to this limited knowledge and the chicken blood's complex matrix, the identification of taste-active and taste-modulating peptides from livestock and poultry blood, chicken blood for example, is challenging.

CN 108047313 A discloses a chicken blood cell antioxidant peptide and a fermentation preparing method. The antioxidant peptide has the amino acid sequence Thr-Ser-Phe-Gly-Asp-Ala-Val-Lys-Asn-Leu-Asp-Asn-Ile-Lys (SEQ ID NO: 1). This antioxidant peptide has a high free radical scavenging activity, high scavenging effect on DPPH free radicals, and high reduction capacity. The method comprises the steps of culture activation, blood cell medium preparation, inoculation fermentation, separation, and purification.

CN 108048518 A discloses a chicken blood cell antioxidant peptide and an enzymolysis preparing method. The antioxidant peptide has the amino acid sequence Met-Gly-Gln-Lys-Asp-Ser-Tyr-Val-Gly-Asp-Glu-Ala-Gln-Ser-Lys-Arg-Gly-Ile-Leu-Thr (SEQ ID NO: 1), or Ala-Glu-Asp-Lys-Lys-Leu-Ile-Gln (SEQ ID NO: 2). These antioxidant peptides have a high free radical scavenging activity, high scavenging effect on DPPH free radicals, superoxide anion free radicals and high reduction capacity. The method comprises the steps of blood cell preparation, hemolysis, proteolysis, and ultrafiltration.

CN 101843289 A discloses a method for producing blood polypeptide protein powder from livestock and poultry blood. According to the method, the large-molecular albumin, which is hard to absorb and utilize in the livestock and poultry blood, is digested into small-molecular albumin, polypeptides, and free amino acids for improving palatability and nutritional value of dried blood for feeding.

Despite developments of products and methods for utilizing livestock and poultry blood mentioned above, there is no disclosure of livestock and poultry blood utilization, specifically the livestock and poultry blood-derived peptides having a specific amino acid sequence, as taste-active and taste-modulating agents.

### SUMMARY OF THE INVENTION

The present invention is invented with the above-mentioned issues in mind.

Therefore, it is an objective of the present invention to provide the utilization of the animal blood, especially livestock and poultry blood, which is an industrial waste from the slaughter process, to obtain new taste-active peptides and a mixture thereof for a taste-modulating agent in food and pharmaceutical preparations.

It is another objective of the present invention to provide animal blood-derived peptides, especially livestock and poultry blood-derived peptides, having specific amino acid sequences. The animal blood-derived peptides of the present invention can be used as taste-active agents that impart taste-modulating effects such as salt-reducing, salt-enhancing, umami, and kokumi effects.

Still another objective of the present invention is to provide a protein hydrolysate and a taste-modulating composition containing the said animal blood-derived peptides.

An additional objective of the present invention is to provide a method for modulating the taste of food and pharmaceutical preparations by using the said animal blood-derived peptides, protein hydrolysate, and taste-modulating composition.

In an aspect, the present invention relates to an isolated peptide having an amino acid sequence selected from the group consisting of: Pro-Val-Leu-Lys (SEQ ID NO: 1); Pro-Leu-Pro-Arg (SEQ ID NO: 2); Glu-Ala-Glu-Phe-Asp (SEQ ID NO: 3); Pro-Glu-Arg-Asn-Glu (SEQ ID NO: 4); Pro-Gln-Pro-Glu-Arg (SEQ ID NO: 5); Glu-Phe-Asp-Glu-Lys-Pro-Ala-Asp (SEQ ID NO: 6); Glu-Asp-Ala-Pro-Val-Leu-His (SEQ ID NO: 7); Glu-Phe-Asp-Ala-Arg-Pro-Thr (SEQ ID NO: 8); Glu-Glu-Thr-Pro-Ser-His-Asp (SEQ ID NO: 9); Glu-Glu-Val-Glu-Glu-Glu-Glu-Val-Glu (SEQ ID NO: 10); Glu-Phe-Asp-Glu-Lys-Ala-Pro-Asp (SEQ ID NO: 11); Glu-Asn-Ala-Pro-Asp-Gln-Lys (SEQ ID NO: 12); Glu-Asp-Met-Ala-Pro (SEQ ID NO: 13); Pro-Ile-Asn-Asp-Asn (SEQ ID NO: 14); Pro-Thr-Asp-Leu (SEQ ID NO: 15); Pro-Val-Ala-Glu (SEQ ID NO: 16); Arg-Asn-Gly-Pro-Arg (SEQ ID NO: 17); Arg-Ala-Glu-Asp-Thr-Ala-Thr-Tyr-Tyr (SEQ ID NO: 18); Glu-Gly-Asp-Ser-Ala-Ala-Ile (SEQ ID NO: 19); Glu-Pro-Glu-Gly-Asp-Val-His-Gln (SEQ ID NO: 20); and Glu-Asp-Glu-Val-Leu-Ala-Thr-Pro (SEQ ID NO: 21).

In another aspect, the present invention relates to a protein hydrolysate comprising at least one peptide having an amino acid sequence selected from the group consisting of: Pro-Val-Leu-Lys (SEQ ID NO: 1); Pro-Leu-Pro-Arg (SEQ ID NO: 2); Glu-Ala-Glu-Phe-Asp (SEQ ID NO: 3); Pro-Glu-Arg-Asn-Glu (SEQ ID NO: 4); Pro-Gln-Pro-Glu-Arg (SEQ ID NO: 5); Glu-Phe-Asp-Glu-Lys-Pro-Ala-Asp (SEQ ID NO: 6); Glu-Asp-Ala-Pro-Val-Leu-His (SEQ ID NO: 7); Glu-Phe-Asp-Ala-Arg-Pro-Thr (SEQ ID NO: 8); Glu-Glu-Thr-Pro-Ser-His-Asp (SEQ ID NO: 9); Glu-Glu-Val-Glu-Glu-Glu-Glu-Val-Glu (SEQ ID NO: 10); Glu-Phe-Asp-Glu-Lys-Ala-Pro-Asp (SEQ ID NO: 11); Glu-Asn-Ala-Pro-Asp-Gln-Lys (SEQ ID NO: 12); Glu-Asp-Met-Ala-Pro (SEQ ID NO: 13); Pro-Ile-Asn-Asp-Asn (SEQ ID NO: 14); Pro-Thr-Asp-Leu (SEQ ID NO: 15); Pro-Val-Ala-Glu (SEQ ID NO: 16); Arg-Asn-Gly-Pro-Arg (SEQ ID NO: 17); Arg-Ala-Glu-Asp-Thr-Ala-Thr-Tyr-Tyr (SEQ ID NO: 18); Glu-Gly-Asp-Ser-Ala-Ala-Ile (SEQ ID NO: 19); Glu-Pro-Glu-Gly-Asp-Val-His-Gln (SEQ ID NO: 20); Glu-Asp-Glu-Val-Leu-Ala-Thr-Pro (SEQ ID NO: 21); and a combination thereof.

In another aspect, the present invention relates to a taste-modulating composition comprising the said isolated peptides or protein hydrolysate as defined above.

In yet another aspect, the present invention relates to a food or pharmaceutical preparation comprising the said isolated peptides, protein hydrolysate, or taste-modulating composition as defined above.

Further, the present invention relates to a method for adjusting the taste of a food or pharmaceutical preparation, comprising applying an effective amount of the said isolated peptides, protein hydrolysate, or taste-modulating composition as defined previously herein to the food or pharmaceutical preparation.

### BRIEF DESCRIPTION OF THE FIGURES

The features of the present invention will be described more clearly from a reading of the following description of an example embodiment. The said example is intended to be illustrative and non-limitation.

FIG. 1 shows the sensory profiles of: (a) low molecular weight (LMW) fractions of hydrolyzed chicken blood cell in model broth (MB) in a concentration of 10 g/L; and (b) LMW fractions of hydrolyzed chicken plasma in model broth in a concentration of 10 g/L.

FIG. 2 shows RP18 MPLC-ELSD chromatogram and comparative taste dilution analysis of MPLC fractions F2-F7 in water (a); in model broth (MB) (b); and in water or model broth (c).

### DETAILED DESCRIPTION OF THE INVENTION

Unless otherwise indicated, any aspects shown herein shall encompass the application to other aspects of the present invention as well.

Unless otherwise specified, technical and scientific terms used herein have the definitions which are understood by a person skilled in the art.

Throughout the present invention, the term “about” is used to indicate that any values shown or presented herein may be varied or deviated. Such variation or deviation may be a result of an error of the equipment or method used to determine the values.

The terms “consist(s) of” and its variation such as “consisting of” and “consisted of”, “comprise(s)” and its variation such as “comprising” and “comprised”, “has/have/having”, “include(s)” and its variation such as “including” and “included” are open-ended verbs. For example, any methods which “consist of”, “comprise”, “have” or “include” one or more components or steps are not limited only to the one or more components or steps, but also cover the components or steps that are not mentioned.

The terms “a”, “an”, “the”, when used to refer to a singular form, are intended to include a plural of that noun as well, unless otherwise specified.

The term “taste-modulating” as used herein is synonymous with the terms “taste-improving”, “taste-adjusting”, “taste-reducing”, taste-enhancing”, and “taste-modifying”, which can be used interchangeably. According to the present invention, the term “taste-modulating agent” and “taste-modulating composition” refer to an agent and a composition capable of imparting one or more tastes, e.g., salty, sweet, kokumi, umami, astringent, bitter, sour, etc., to food or pharmaceutical preparation and able to modify the taste of the food or pharmaceutical preparation as desired. In addition, the taste adjustment can be obtained by reducing or enhancing one or more of those tastes.

The term “food preparation” as used herein refers to all edible food and beverage products, compositions, and formulations for humans and animals, including meals, drinks, dietary supplements, snacks, desserts, candies, for examples. Similarly, the term “pharmaceutical preparation” as used herein refers to all edible pharmaceutical products, compositions, and formulations. The food and pharmaceutical preparations can be in the form of solids, e.g., powders, tablets, granules, beads, capsules, etc., semi-solids, gels, and liquids, for examples.

Any tools, equipment, methods, materials, or chemicals mentioned herein, unless otherwise indicated, mean the tool, equipment, methods, materials, or chemicals generally used or practiced by a person skilled in the art.

All disclosed chemicals, compounds, materials, components and/or methods and the claims of the present invention are intended to cover the aspects of the invention obtained from any actions, practices, modifications or changes made to the factors without conducting significantly different experiment from the present invention, and to obtain an object with properties, utilities and effects that are similar to the aspects of the present invention according to the opinions of a person having ordinary skill in the art, although not particularly indicated in the claims. Therefore, the object that is equivalent or similar to the aspects of the present invention, including any slight modification or change apparent to a person of ordinary skill in the art, should also be considered to be within the spirit, scope, and concept of the present invention.

Hereinafter, the present invention will be described in more detail.

According to the first aspect of the present invention, the isolated peptides have amino acid sequence selected from the group consisting of: Pro-Val-Leu-Lys (SEQ ID NO: 1); Pro-Leu-Pro-Arg (SEQ ID NO: 2); Glu-Ala-Glu-Phe-Asp (SEQ ID NO: 3); Pro-Glu-Arg-Asn-Glu (SEQ ID NO: 4); Pro-Gln-Pro-Glu-Arg (SEQ ID NO: 5); Glu-Phe-Asp-Glu-Lys-Pro-Ala-Asp (SEQ ID NO: 6); Glu-Asp-Ala-Pro-Val-Leu-His (SEQ ID NO: 7); Glu-Phe-Asp-Ala-Arg-Pro-Thr (SEQ ID NO: 8); Glu-Glu-Thr-Pro-Ser-His-Asp (SEQ ID NO: 9); Glu-Glu-Val-Glu-Glu-Glu-Glu-Val-Glu (SEQ ID NO: 10); Glu-Phe-Asp-Glu-Lys-Ala-Pro-Asp (SEQ ID NO: 11); Glu-Asn-Ala-Pro-Asp-Gln-Lys (SEQ ID NO: 12); Glu-Asp-Met-Ala-Pro (SEQ ID NO: 13); Pro-Ile-Asn-Asp-Asn (SEQ ID NO: 14); Pro-Thr-Asp-Leu (SEQ ID NO: 15); Pro-Val-Ala-Glu (SEQ ID NO: 16); Arg-Asn-Gly-Pro-Arg (SEQ ID NO: 17); Arg-Ala-Glu-Asp-Thr-Ala-Thr-Tyr-Tyr (SEQ ID NO: 18); Glu-Gly-Asp-Ser-Ala-Ala-Ile (SEQ ID NO: 19); Glu-Pro-Glu-Gly-Asp-Val-His-Gln (SEQ ID NO: 20); and Glu-Asp-Glu-Val-Leu-Ala-Thr-Pro (SEQ ID NO: 21).

The isolated peptides of the present invention can impart at least one of the following effects: salt-reducing, salt-enhancing, umami, and kokumi effects.

In a preferred aspect, the isolated peptides which impart the salt-reducing effect have the amino acid sequence selected from the group consisting of: Pro-Val-Leu-Lys (SEQ ID NO: 1); Glu-Ala-Glu-Phe-Asp (SEQ ID NO: 3); and Glu-Asp-Met-Ala-Pro (SEQ ID NO: 13).

In another preferred aspect, the isolated peptides which impart the salt-enhancing effect have the amino acid sequence selected from the group consisting of: Glu-Phe-Asp-Glu-Lys-Pro-Ala-Asp (SEQ ID NO: 6); Glu-Phe-Asp-Ala-Arg-Pro-Thr (SEQ ID NO: 8); Glu-Phe-Asp-Glu-Lys-Ala-Pro-Asp (SEQ ID NO: 11); Pro-Ile-Asn-Asp-Asn (SEQ ID NO: 14); Pro-Val-Ala-Glu (SEQ ID NO: 16); and Glu-Asp-Glu-Val-Leu-Ala-Thr-Pro (SEQ ID NO: 21).

In a further preferred aspect, the isolated peptides which impart the umami effect have the amino acid sequence selected from the group consisting of: Pro-Val-Leu-Lys (SEQ ID NO: 1); Pro-Leu-Pro-Arg (SEQ ID NO: 2); Glu-Ala-Glu-Phe-Asp (SEQ ID NO: 3); Pro-Glu-Arg-Asn-Glu (SEQ ID NO: 4); Pro-Gln-Pro-Glu-Arg (SEQ ID NO: 5); Glu-Phe-Asp-Glu-Lys-Pro-Ala-Asp (SEQ ID NO: 6); Glu-Asp-Ala-Pro-Val-Leu-His (SEQ ID NO: 7); Glu-Glu-Thr-Pro-Ser-His-Asp (SEQ ID NO: 9); Glu-Glu-Val-Glu-Glu-Glu-Glu-Val-Glu (SEQ ID NO: 10); Glu-Phe-Asp-Glu-Lys-Ala-Pro-Asp (SEQ ID NO: 11); Glu-Asn-Ala-Pro-Asp-Gln-Lys (SEQ ID NO: 12); Pro-Ile-Asn-Asp-Asn (SEQ ID NO: 14); Arg-Asn-Gly-Pro-Arg (SEQ ID NO: 17); Arg-Ala-Glu-Asp-Thr-Ala-Thr-Tyr-Tyr (SEQ ID NO: 18); Glu-Gly-Asp-Ser-Ala-Ala-Ile (SEQ ID NO: 19); Glu-Pro-Glu-Gly-Asp-Val-His-Gln (SEQ ID NO: 20); and Glu-Asp-Glu-Val-Leu-Ala-Thr-Pro (SEQ ID NO: 21).

In a still further preferred aspect, the isolated peptides which impart the kokumi effect have the amino acid sequence selected from the group consisting of: Pro-Val-Leu-Lys (SEQ ID NO: 1); Pro-Leu-Pro-Arg (SEQ ID NO: 2); Glu-Ala-Glu-Phe-Asp (SEQ ID NO: 3); Pro-Glu-Arg-Asn-Glu (SEQ ID NO: 4); Pro-Gln-Pro-Glu-Arg (SEQ ID NO: 5); Glu-Asp-Ala-Pro-Val-Leu-His (SEQ ID NO: 7); Glu-Phe-Asp-Ala-Arg-Pro-Thr (SEQ ID NO: 8); Glu-Glu-Thr-Pro-Ser-His-Asp (SEQ ID NO: 9); Glu-Glu-Val-Glu-Glu-Glu-Glu-Val-Glu (SEQ ID NO: 10); Glu-Phe-Asp-Glu-Lys-Ala-Pro-Asp (SEQ ID NO: 11); Glu-Asn-Ala-Pro-Asp-Gln-Lys (SEQ ID NO: 12); Glu-Asp-Met-Ala-Pro (SEQ ID NO: 13); Pro-Thr-Asp-Leu (SEQ ID NO: 15); Pro-Val-Ala-Glu (SEQ ID NO: 16); Arg-Asn-Gly-Pro-Arg (SEQ ID NO: 17); Arg-Ala-Glu-Asp-Thr-Ala-Thr-Tyr-Tyr (SEQ ID NO: 18); Glu-Gly-Asp-Ser-Ala-Ala-Ile (SEQ ID NO: 19); Glu-Pro-Glu-Gly-Asp-Val-His-Gln (SEQ ID NO: 20); and Glu-Asp-Glu-Val-Leu-Ala-Thr-Pro (SEQ ID NO: 21).

The isolated peptides of the present invention are obtained or derived from enzymatic hydrolysis or fermentation. Specifically, the isolated peptides are obtained or derived from the enzymatic hydrolysis of animal blood including, but not limited to pigs, cows, and poultry such as chickens, birds, turkeys, etc. In a preferred aspect, the animal blood useful for the present invention is poultry blood, more preferably chicken blood.

In an exemplary embodiment, the enzymatic hydrolysis for obtaining the isolated peptides of the present invention is carried out by using serine protease. Preferably, the serine protease is subtilisin.

The present invention also provides the use of the isolated peptides as previously defined as a taste-modulating agent for a food or pharmaceutical preparation.

The second aspect of the present invention relates to a protein hydrolysate comprising at least one peptide having an amino acid sequence selected from the group consisting of: Pro-Val-Leu-Lys (SEQ ID NO: 1); Pro-Leu-Pro-Arg (SEQ ID NO: 2); Glu-Ala-Glu-Phe-Asp (SEQ ID NO: 3); Pro-Glu-Arg-Asn-Glu (SEQ ID NO: 4); Pro-Gln-Pro-Glu-Arg (SEQ ID NO: 5); Glu-Phe-Asp-Glu-Lys-Pro-Ala-Asp (SEQ ID NO: 6); Glu-Asp-Ala-Pro-Val-Leu-His (SEQ ID NO: 7); Glu-Phe-Asp-Ala-Arg-Pro-Thr (SEQ ID NO: 8); Glu-Glu-Thr-Pro-Ser-His-Asp (SEQ ID NO: 9); Glu-Glu-Val-Glu-Glu-Glu-Glu-Val-Glu (SEQ ID NO: 10); Glu-Phe-Asp-Glu-Lys-Ala-Pro-Asp (SEQ ID NO: 11); Glu-Asn-Ala-Pro-Asp-Gln-Lys (SEQ ID NO: 12); Glu-Asp-Met-Ala-Pro (SEQ ID NO: 13); Pro-Ile-Asn-Asp-Asn (SEQ ID NO: 14); Pro-Thr-Asp-Leu (SEQ ID NO: 15); Pro-Val-Ala-Glu (SEQ ID NO: 16); Arg-Asn-Gly-Pro-Arg (SEQ ID NO: 17); Arg-Ala-Glu-Asp-Thr-Ala-Thr-Tyr-Tyr (SEQ ID NO: 18); Glu-Gly-Asp-Ser-Ala-Ala-Ile (SEQ ID NO: 19); Glu-Pro-Glu-Gly-Asp-Val-His-Gln (SEQ ID NO: 20); Glu-Asp-Glu-Val-Leu-Ala-Thr-Pro (SEQ ID NO: 21); and a combination thereof.

In a preferred embodiment, the protein hydrolysate of the present invention comprises at least the peptides having the amino acid sequence of: Glu-Ala-Glu-Phe-Asp (SEQ ID NO: 3); Glu-Phe-Asp-Glu-Lys-Pro-Ala-Asp (SEQ ID NO: 6); Glu-Phe-Asp-Glu-Lys-Ala-Pro-Asp (SEQ ID NO: 11); Pro-Ile-Asn-Asp-Asn (SEQ ID NO: 14); and Pro-Thr-Asp-Leu (SEQ ID NO: 15).

The protein hydrolysate of the present invention can be obtained or derived by enzymatic hydrolysis. Particularly, the protein hydrolysate is obtained by the enzymatic hydrolysis of animal blood including, but not limited to pigs, cows, and poultry such as chickens, birds, turkeys, etc. In a preferred aspect, the animal blood is poultry blood. It is more preferred that the poultry blood is chicken blood.

The enzymatic hydrolysis for obtaining the protein hydrolysate of the present invention is carried out by using serine protease. Preferably, the serine protease is subtilisin.

The invention also provides the use of the protein hydrolysate as defined previously herein as a taste-modulating agent for a food or pharmaceutical preparation.

The third aspect of the present invention relates to a taste-modulating composition comprising the said isolated peptides or protein hydrolysate as previously defined. The invention also provides the use of the taste-modulating composition in a food or pharmaceutical preparation. Therefore, the present invention provides a food or pharmaceutical preparation comprising the isolated peptides, protein hydrolysate or taste-modulating composition as previously defined.

In addition, the present invention provides a method for adjusting the taste of a food preparation, comprising applying an effective amount of the said isolated peptides, protein hydrolysate or taste-modulating composition which comprises an effective amount of the isolated peptides to the food or pharmaceutical preparation.

### EXAMPLE

Hereinafter, the present invention will be described in more detail by the following non-limiting examples.

### Example 1: Preparation and Identification of Peptides

### 1. Preparation of Peptides by Enzymatic Digestion

All samples were prepared by using chicken blood from a slaughterhouse. Firstly, the chicken blood was centrifuged at 4000 rpm for 8 min and then digested with subtilisin enzyme (Protin SD-AY10) using 2M NaOH to adjust the initial pH to 9. The enzyme-digested samples were incubated at 55° C. for 18 h at 120 rpm. Afterward, the enzyme activity was stopped by heating in a water bath at 100° C. for 15 min and cooling down to an ambient temperature. The obtained solutions were freeze-dried and stored at −20° C. until further use.

### 2. Fractionation of Peptide Mixture by Ultrafiltration Membrane

5 g of chicken blood plasma powder was dissolved in 300 mL deionized water in an ultrasonic bath. The obtained solution was transferred to an ultrafiltration system (8400 Millipore Amicon, EMD Millipore) equipped with a cut-off membrane of 1, 3 or 5 kDa, respectively (Ultacel® chemistry regenerated cellulose (RC) filter of 76 mm in diameter, EMD Millipore corporation, Billerica, USA). The high molecular weight (HMW) and low molecular weight (LMW) fractions of each cut-off were washed with 50 mL deionized water, freeze-dried, and stored at −20° C. until further analysis. The yields of different membrane cut-offs are summarized in Table 1 and highlight that the LMW fractions of each cut-off had the highest contents of compounds.

TABLE 1

| Cut-off membrane | Sample | Yield (%) |
|---|---|---|
| 1 kDa | LMW blood cell | 60.5 |
| | HMW blood cell | 13.6 |
| | LMW plasma | 75.2 |
| | HMW plasma | 10.1 |
| 3 kDa | LMW blood cell | 61.1 |
| | HMW blood cell | 34.7 |
| | LMW plasma | 75.8 |
| | HMW plasma | 20.1 |
| 5 kDa | LMW blood cell | 61.8 |
| | HMW blood cell | 29.1 |
| | LMW plasma | 78.4 |
| | HMW plasma | 12.1 |

To check which of LMW fractions contain the highest content of taste-active and taste-modulating peptides, the sodium and chloride content analysis of all LMW fractions of blood cell and plasma was performed prior to the sensory evaluation, and liquid chromatography with tandem mass spectrometry (LC-MS/MS). The quantitative analysis by ion chromatography indicated that chicken blood does not contain high contents of sodium and chloride since none of the samples showed more than 10% sodium and chloride (Table 2). The plasma samples contained more sodium and chloride than blood cells and the contents of sodium and chloride of 1 kDa LMW plasma were the highest among all samples.

TABLE 2

| Sample | Sodium content (%) | Chloride content (%) |
|---|---|---|
| 1 kDa LMW blood cell | 1.5 | 0.9 |
| 1 kDa LMW plasma | 5.1 | 6.4 |
| 3 kDa LMW blood cell | 1.1 | 0.5 |
| 3 kDa LMW plasma | 4.2 | 6.7 |
| 5 kDa LMW blood cell | 1.5 | 0.3 |
| 5 kDa LMW plasma | 4.3 | 6.2 |

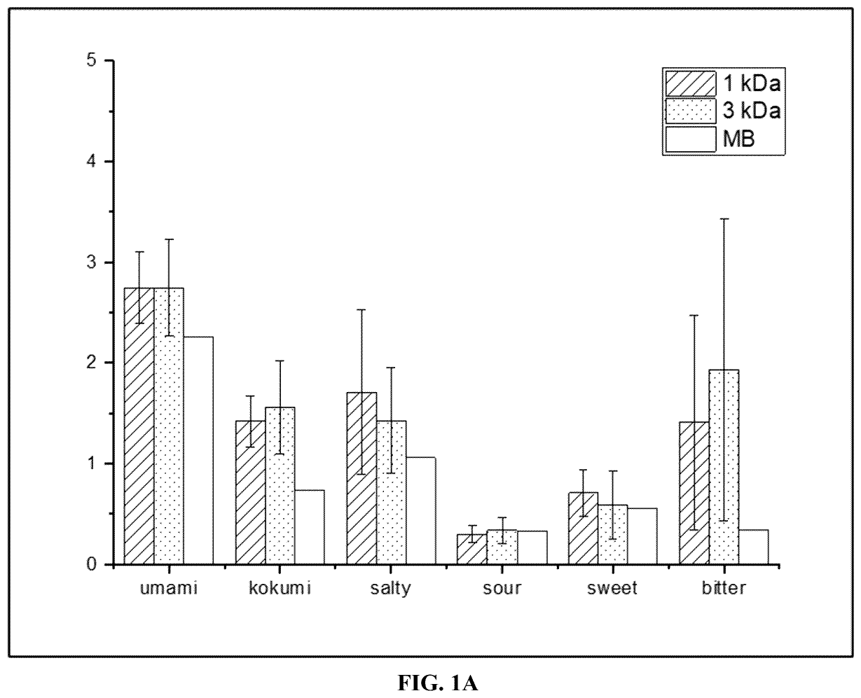

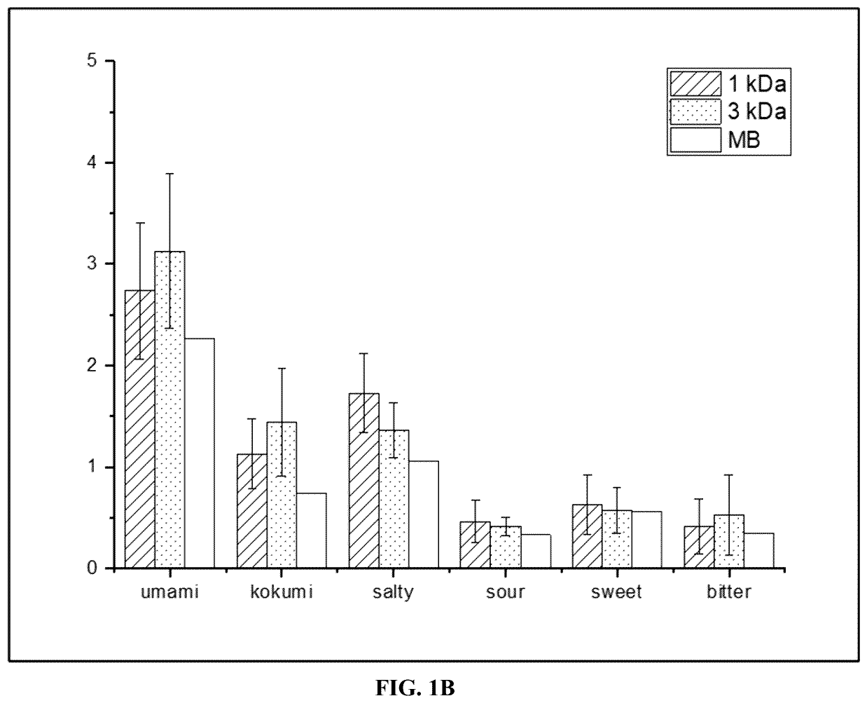

The sensory evaluation was designed to get a clear and comprehensive understanding of samples' taste properties. The HMW fraction was not used for sensory experiments due to safety reasons. The sensory samples were prepared by dissolving each LMW fraction of blood cell and plasma in model broth to a final concentration of 10 g/L and the pH was adjusted to 6 by adding 1% formic acid in water. Panelists were asked to evaluate six taste qualities including umami, kokumi, sweet, salty, sour, and bitter on a score from 0 (not detectable) to 5 (strongly detectable) as shown in FIGS. 1(a) and (b).

From experimental results, it was found that LMW of 3 kDa cut-off hydrolyzed chicken blood cell has significantly (P<0.01) more taste-active and taste-modulating properties with respect to kokumi (3 kDa: 1.6), sweetness (3 kDa: 0.6), and bitterness (3 kDa: 2.0) than 1 kDa. Saltiness and sweetness were adverse. The umami enhancement was similar for both 1 kDa and 3 kDa cut-offs. In addition, LMW fractions of 3 kDa and 5 kDa cut-offs were compared using duo-trio tests for both blood cell and plasma. The sensory test was performed in two series, AAB and BBA (order was random). Panelists were asked to choose the sample which was different from the other two samples in every set. Duo-trio test for comparison between LMW fractions of hydrolyzed blood cell of 3 kDa and plasma of 3 kDa was designed to choose the sample with more pronounced taste compared to the other two samples. From experimental results, there was no significant difference (P=0.1) which indicated that peptides larger than 3 kDa did not exhibit significant taste-active and taste-modulating activity. 3 kDa LMW plasma showed more pronounced umami, kokumi, and salty impression compared to LMW fraction of blood cell. Therefore, 3 kDa cut-off LMW plasma fraction was chosen for further investigation.

### 3. Fractionation of 3 kDa LMW Plasma by Medium Pressure Liquid Chromatography (MPLC)

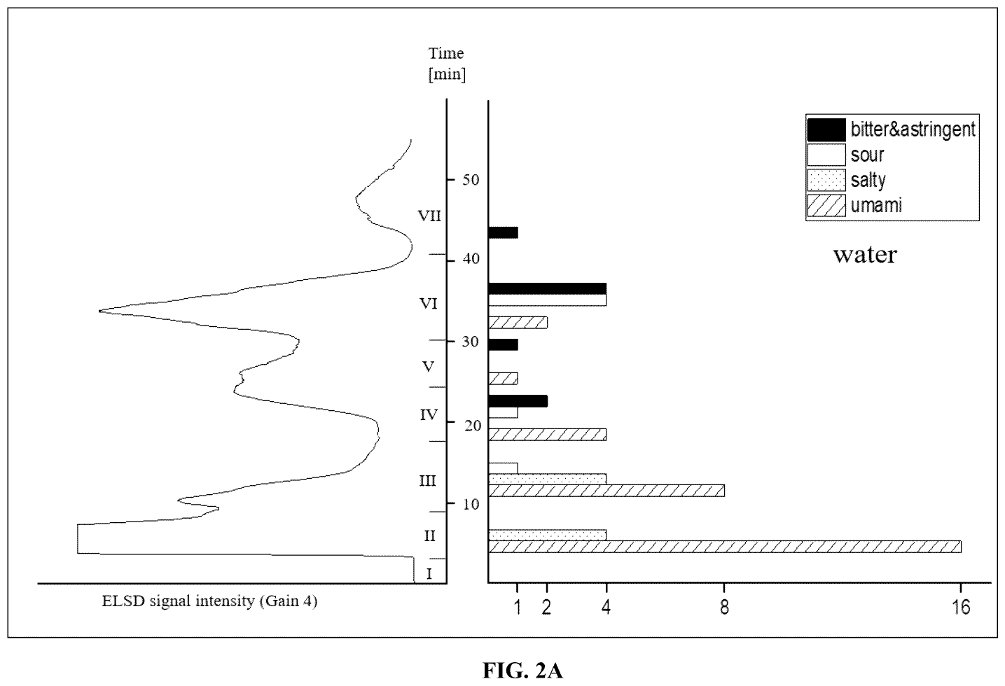

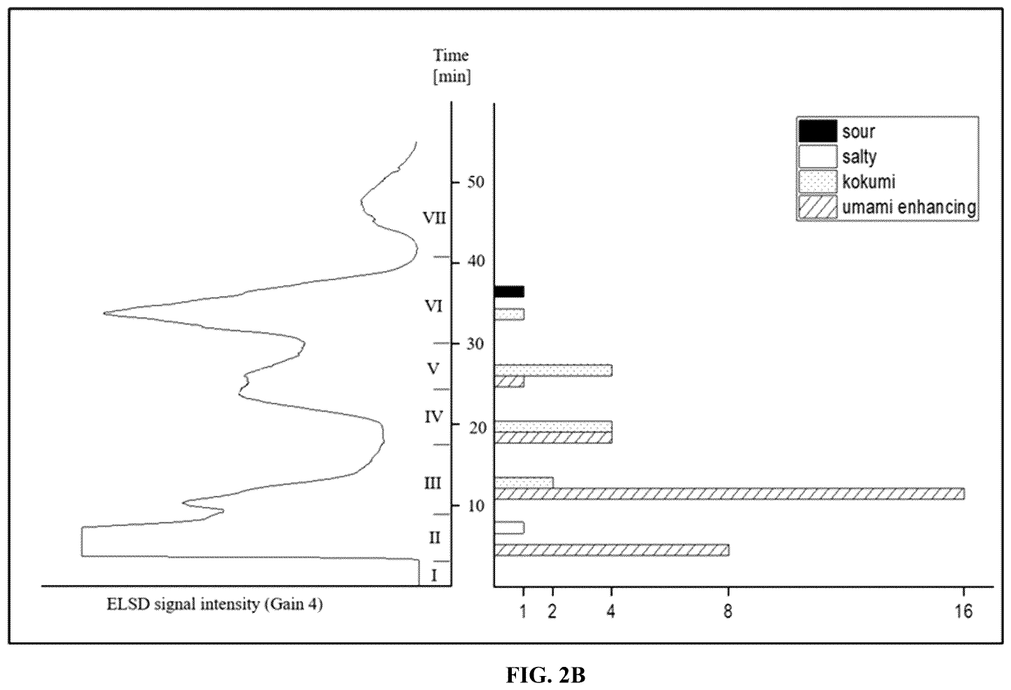

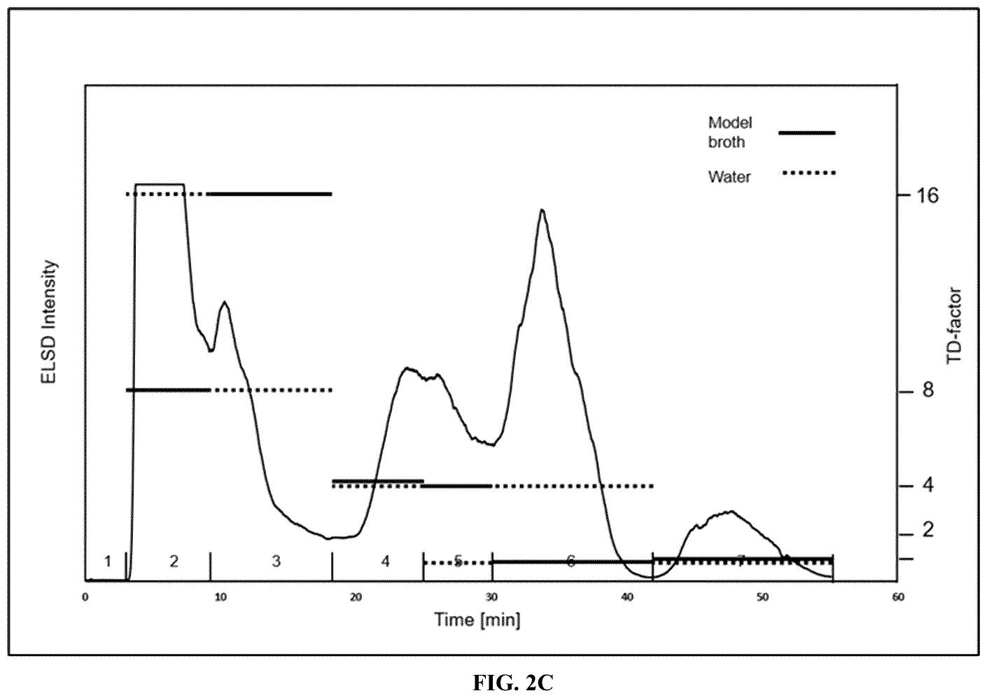

2 g of 3 kDa LMW plasma powder was dissolved in 20 mL deionized water and fractionated by means of reversed phase (RP) MPLC (Büchi, Flawil, Switzerland) on a 150×40 mm polypropylene cartridge using 25-40 μm LiChroPrep RP18 bulk material (Merck, Darmstadt, Germany) as stationary phase and a gradient of 1% aqueous formic acid (solvent A) and methanol (solvent B) as mobile phase. The separation was monitored using an evaporative light scattering detector (ELSD) (SEDERE, Alfortville Cedex, France) and chromatography (flow rate of 40 mL/min) was performed with 100% solvent A for 10 min and then solvent B was increased to 30% within 15 min and to 100% within additional 15 min, followed by 15 min elution with 100% solvent B. Finally, seven fractions (F1-F7) were collected, separated from the solvent in vacuum, and lyophilized twice prior to taste dilution analysis (TDA), comparative taste dilution analysis (cTDA) and chemical analysis (FIGS. 2(a), (b) and (c)).

TDA was performed to locate the most intense taste-active fraction and cTDA was performed to locate the taste-modulating fraction. For TDA, serial 1:2 dilutions of each fraction or subfraction were prepared in water and then presented to sensory panelists in order of increasing concentrations. Each dilution was evaluated sensorially using the duo-trio test. The dilution at which a taste difference between the diluted fraction and two blanks (water) could just be detected was defined as the taste dilution (TD) factor. The TD factor evaluated by five different panelists was averaged. The TD factor between individuals and three separate sessions differed by not more than one dilution step. Eight dilution steps were performed (Oliver Frank, Jezussek, & Hofmann, 2003). For cTDA, the procedure was as described for TDA except that the fractions were dissolved in model broth and the model broth was used as blanks (Dunkel et at., 2007; Salger et al., 2019). The comparative taste dilution (cTD) factor is defined by the dilution at which a taste difference between the diluted fraction and two blanks (model broth) could just be detected.

According to TDA and cTDA results, fraction F3 exhibited the highest taste-active and taste-modulating properties. In water, fraction F2 showed the highest intrinsic umami taste with TD factor of 16, followed by fraction F3 (TD factor=8) and fraction F4 (TD factor=4) as shown in FIG. 2(a). For salty taste, fractions F2 and F3 have identical TD factors of 4. Fraction F6 exhibited the highest bitter and astringent taste (TD factor=4). In model broth, fraction F3 exhibited the highest umami-enhancing activity (cTD factor=16), followed by fraction F2 (cTD factor=8) and fraction F4 (cTD factor=4) as shown in FIG. 2(b). The highest kokumi taste was found for fraction F4. None of the fractions elicited any bitter taste in model broth highlighting the potential of fractions F2-F5 as taste-enhancing fractions without unpleasant intrinsic taste (FIG. 2(c)). As fractions F2, F3 and F4 had the highest TD and cTD factors, the further analysis focused on these factions.

### 4. Subfractionation of Fractions F2, F3, and F4 by High Performance Liquid Chromatography (HPLC)

To identify the taste-active and taste-modulating peptides in the fractions F2, F3, and F4, these fractions were further separated by means of preparative HPLC with a gradient of 1% aqueous formic acid solution and acetonitrile.

For separation of fraction F2, 10 g of fraction F2 was dissolved in 1 L deionized water and membrane-filtered (0.45 μm). 0.5 mL of this solution was injected into the HPLC system (Jasco, Groß-Umstadt, Germany). The HPLC separation was performed under the following conditions.
Column: Luna HILIC (200 A, 250×21.2 mm, 5 μm)
Flow rate: 18 mL/min
Detector: ELSD and UV (254 nm)

For separation of fraction F3, 10 g of fraction F3 was dissolved in 1 L deionized water and membrane-filtered (0.45 μm). 1 mL of this solution was injected into the HPLC system (Jasco, Groß-Umstadt, Germany). The HPLC separation was performed under the following conditions.
Column: Luna PFP (2) (100 A, 250×21.2 mm, 5 μm)
Flow rate: 21 mL/min
Detector: ELSD and UV (254 nm)

For separation of fraction F4, 20 g of fraction F4 was dissolved in 1 L deionized water and membrane-filtered (0.45 μm). 1 mL of this solution was injected into the HPLC system (Jasco, Groß-Umstadt, Germany). The HPLC separation was performed under the following conditions.
Column: Nucleodur C18 Pyramid (100 A, 250×21.2 mm, 5 μm)
Flow rate: 20 mL/min
Detector: ELSD and UV (254 nm)

The subfractions of F2, F3, and F4 were analyzed using liquid chromatography time-of-flight mass spectrometry (LC-TOF-MS) analysis using a C8 column and peptides were identified using PEAKS software.

From the experimental results, the fractions F3 and F4 were chosen for large-scale separation to identify novel taste-active and taste-modulating peptides since the fraction F2 contained a high concentration of already-known taste-active and taste-modulating molecules (e.g., cations, amino acids, and glutamyl dipeptides).

### Example 2: Identification and Evaluation of Taste Active and Taste-Modulating Effect of Peptides

### 1. Identification of Peptides

After analyzing the HPLC subfractions of MPLC fractions F2, F3, and F4, the potential taste-active and taste-modulating peptides present in the fractions F3 and F4 were identified by using the combination of proteomics and sensomics techniques (the so-called sensoproteomics approach) which is adapted from the method represented by Sebald et al., J. Agric. Food Chem. 2018, 66, 11092-11104. Targeted and untargeted proteomics are both applied to improve productiveness of the present invention.

By using targeted proteomics methods, all the theoretically possible peptides from chicken serum albumin and corticosteroid binding globulin were investigated and 767 peptides were detected. Untargeted proteomics using Maxquant and PEAKS softwares was followed to identify peptides in LC-TOF-MS data of the subfractions of MPLC fractions F2, F3, and F4. All peptides that were identified in fractions F3 and F4 using de novo sequencing and database search in PEAKS with an ALC (average local confidence)>80 and with a N-terminal E, P or R were selected. By using Maxquant, taste-active and taste-modulating fractions were investigated first and then the identified peptides were sorted according to a N-terminal E, Por R and the peak areas. 11 peptides from fraction F3 and 44 peptides from fraction F4 were further confirmed by MS² data.

After the identification of peptides followed by several filtering steps, peptide candidates were chosen for validation. Final peptide candidates identified in MPLC fractions F3 and F4 are shown in Table 3 below.

TABLE 3

| | | | | | Area | ALC |
|---|---|---|---|---|---|---|
| | | | Product | Identified | (PEAKS) or | (PEAKS) or |
| | Peptide | RT | ion | by means | intensity | score |
| Fraction | sequence | (min) | confidence | of | (MaxQuant) | (MaxQuant) |
| F4 | Pro-Val- | 11.82 | 5 | de novo | 2.19 × 10⁵ | 97 |
| | Leu-Lys | | | | | |
| | (SEQ ID | | | | | |
| | NO: 1) | | | | | |
| F4 | Pro-Leu- | 9.07 | 5 | de novo | 1.73 × 10⁴ | 92 |
| | Pro-Arg | | | | | |
| | (SEQ ID | | | | | |
| | NO: 2) | | | | | |
| F4 | Glu-Ala- | 4.65 | 5 | database | 2048.6 | 80.317 |
| | Glu-Phe- | | | | | |
| | Asp (SEQ | | | | | |
| | ID NO: 3) | | | | | |
| F3 and | Pro-Glu- | 0.98(F3), 1.23(F4) | 5 | de novo | 3.35 × 10⁴(F3), | 98 |
| F4 | Arg-Asn- | | | | 3.57 × 10⁴(F4) | |
| | Glu (SEQ | | | | | |
| | ID NO: 4) | | | | | |
| F3 | Pro-Gln- | 1.03 | 5 | de novo | 2.86 × 10³ | 99 |
| | Pro-Glu-Arg | | | | | |
| | (SEQ ID | | | | | |
| | NO: 5) | | | | | |
| F4 | Glu-Phe- | 4.18 | 5 | Database | 1.49 × 10⁵ | 95 |
| | Asp-Glu- | | | and | | |
| | Lys-Pro- | | | de novo | | |
| | Ala-Asp | | | | | |
| | (SEQ ID | | | | | |
| | NO: 6) | | | | | |
| F4 | Glu-Asp- | 10.979 | 5 | de novo | 1.84 × 10⁴ | 98 |
| | Ala-Pro- | | | | | |
| | Val-Leu-His | | | | | |
| | (SEQ ID | | | | | |
| | NO: 7) | | | | | |
| F4 | Glu-Phe- | 4.18 | 4 | de novo | 7.13 × 10⁴ | 80 |
| | Asp-Ala- | | | | | |
| | Arg-Pro-Thr | | | | | |
| | (SEQ ID | | | | | |
| | NO: 8) | | | | | |
| F4 | Glu-Glu- | 1.23 | 4 | de novo | 1.33 × 10⁴ | 87 |
| | Thr-Pro-Ser- | | | | | |
| | His-Asp | | | | | |
| | (SEQ ID | | | | | |
| | NO: 9) | | | | | |
| F3 | Glu-Glu- | 1 | 4 | database | 1.06 × 10² | — |
| | Val-Glu- | | | and | | |
| | Glu-Glu- | | | de novo | | |
| | Glu-Val-Glu | | | | | |
| | (SEQ ID | | | | | |
| | NO: 10) | | | | | |
| F4 | Glu-Phe- | 4.18 | 5 | de novo | 1.49 × 10⁵ | 94 |
| | Asp-Glu- | | | | | |
| | Lys-Ala- | | | | | |
| | Pro-Asp | | | | | |
| | (SEQ ID | | | | | |
| | NO: 11) | | | | | |
| F4 | Glu-Asn- | 1.23 | 5 | database | 1.70 × 10⁴ | 98 |
| | Ala-Pro- | | | and | | |
| | Asp-Gln- | | | de novo | | |
| | Lys (SEQ | | | | | |
| | ID NO: 12) | | | | | |
| F4 | Glu-Asp- | 4.12 | 5 | database | 3787.1 | 108.73 |
| | Met-Ala-Pro | | | | | |
| | (SEQ ID | | | | | |
| | NO: 13) | | | | | |
| F4 | Pro-Ile-Asn- | 2.08 | 5 | database | 32311 | 107.73 |
| | Asp-Asn | | | | | |
| | (SEQ ID | | | | | |
| | NO: 14) | | | | | |
| F4 | Pro-Thr- | 4.49 | 5 | database | 906800 | 115.49 |
| | Asp-Leu | | | | | |
| | (SEQ ID | | | | | |
| | NO: 15) | | | | | |
| F4 | Pro-Val- | 2.15 | 4 | database | 375570 | 109.97 |
| | Ala-Glu | | | | | |
| | (SEQ ID | | | | | |
| | NO: 16) | | | | | |
| F3 | Arg-Asn- | 0.87 | 4 | de novo | 3.77 × 10³ | 99 |
| | Gly-Pro-Arg | | | | | |
| | (SEQ ID | | | | | |
| | NO: 17) | | | | | |
| F4 | Arg-Ala- | 13.5 | 5 | database | 5940 | — |
| | Glu-Asp- | | | | | |
| | Thr-Ala- | | | | | |
| | Thr-Tyr-Tyr | | | | | |
| | (SEQ ID | | | | | |
| | NO: 18) | | | | | |
| F4 | Glu-Gly- | 3.2 | 4 | database | 64600 | — |
| | Asp-Ser- | | | | | |
| | Ala-Ala-Ile | | | | | |
| | (SEQ ID | | | | | |
| | NO: 19) | | | | | |
| F4 | Glu-Pro- | 1.23 | 4 | de novo | 1.45 × 10⁴ | 87 |
| | Glu-Gly- | | | | | |
| | Asp-Val- | | | | | |
| | His-Gln | | | | | |
| | (SEQ ID | | | | | |
| | NO: 20) | | | | | |
| F4 | Glu-Asp- | 5.92 | 4 | de novo | 3.44 × 10⁴ | 80 |
| | Glu-Val- | | | | | |
| | Leu-Ala- | | | | | |
| | Thr-Pro | | | | | |
| | (SEQ ID | | | | | |
| | NO: 21) | | | | | |

The 21 candidate peptides were synthesized, and their purities were evaluated by means of quantitative ¹H-NMR ranging from 62% to 100%. The presence of the peptides in the samples was checked by means of LC-MS/MS using Skyline. Comparing the MRM (Multiple Reaction Monitoring) transitions detected in MPLC fractions F3 or F4 with the synthesized peptides, it could be confirmed that the candidate peptides exist in the hydrolyzed chicken plasma according to the present invention.

### 2. Determination of Taste Thresholds of Peptides

The threshold concentrations of 21 newly identified peptides were determined in water for the intrinsic taste and in model broth (pH 6.0 adjusted with 1% formic acid) for the taste modulating properties, respectively (Table 4). The peptides were dissolved, serially diluted 1:2, and presented to the panel using duo-trio-tests with ascending concentrations as reported in literatures. The geometric mean of the last and the second to last concentration was calculated and taken as the individual recognition threshold. The threshold value of the sensory panel was approximated by averaging the threshold values of the individuals in two independent sessions.

$${C}_{p} = \sqrt{Co \times C 1}$$

wherein
Cₚ: the threshold of a panelist,
C₁: the first correctly evaluated concentration,
C₀: the first misjudged concentration

$${C}_{s} = \sqrt[n]{{\prod }_{i = 1}^{n} Cpj}$$

wherein
Cₛ: the thresholds of total panelists,
Cₚ: the threshold of a panelist,
n: the number of panel members

TABLE 4

| | Threshold in | Taste | Threshold | Taste |
|---|---|---|---|---|
| | model broth | properties in | in water | properties |
| Peptide sequence | (μmol/L) | model broth | (μmol/L) | in water |
| Pro-Val-Leu-Lys | 296 | kokumi, salty, | tasteless | — |
| (SEQ ID NO: 1) | | umami, | | |
| | | astringent | | |
| Pro-Leu-Pro-Arg | 112 | umami, kokumi | 296 | astringent |
| (SEQ ID NO: 2) | | | | |
| Glu-Ala-Glu-Phe- | 95 | kokumi, salty, | 116 | astringent, |
| Asp | | umami | | bitter |
| (SEQ ID NO: 3) | | | | |
| Pro-Glu-Arg-Asn- | 231 | umami, kokumi, | 100 | astringent, |
| Glu | | sweet. bitter, | | sweet |
| (SEQ ID NO: 4) | | sour | | |
| Pro-Gln-Pro-Glu- | 133 | umami, kokumi, | 705 | astringent, |
| Arg | | astringent | | bitter |
| (SEQ ID NO: 5) | | | | |
| Glu-Phe-Asp-Glu- | 94 | mouthfeel, | tasteless | — |
| Lys-Pro-Ala-Asp | | sweet, umami, | | |
| (SEQ ID NO: 6) | | astringent | | |
| Glu-Asp-Ala-Pro- | 236 | kokumi, | 565 | astringent |
| Val-Leu-His | | umami, bitter, | | |
| (SEQ ID NO: 7) | | mouthfeel | | |
| Glu-Phe-Asp-Ala- | 351 | kokumi, salty | tasteless | — |
| Arg-Pro-Thr | | | | |
| (SEQ ID NO: 8) | | | | |
| Glu-Glu-Thr-Pro- | 36 | umami, kokumi | 283 | astringent, |
| Ser-His-Asp | | | | sour |
| (SEQ ID NO: 9) | | | | |
| Glu-Glu-Val-Glu- | 34 | umami, kokumi | 233 | bitter, |
| Glu-Glu-Glu-Val- | | | | astringent |
| Glu | | | | |
| (SEQ ID NO: 10) | | | | |
| Glu-Phe-Asp-Glu- | 67 | kokumi, umami, | 865 | bitter |
| Lys-Ala-Pro-Asp | | less salty | | |
| (SEQ ID NO: 11) | | | | |
| Glu-Asn-Ala-Pro- | 137 | kokumi, umami | 907 | bitter, |
| Asp-Gln-Lys | | | | umami |
| (SEQ ID NO: 12) | | | | |
| Glu-Asp-Met-Ala- | 151 | kokumi, salty | 1024 | bitter, |
| Pro | | | | astringent |
| (SEQ ID NO: 13) | | | | |
| Pro-Ile-Asn-Asp- | 26 | umami, salty, | tasteless | — |
| Asn | | mouthful | | |
| (SEQ ID NO: 14) | | | | |
| Pro-Thr-Asp-Leu | 97 | kokumi, | tasteless | — |
| (SEQ ID NO: 15) | | mouthfeel | | |
| Pro-Val-Ala-Glu | 243 | kokumi, salty | 688 | astringent, |
| (SEQ ID NO: 16) | | | | sour |
| Arg-Asn-Gly-Pro- | 101 | kokumi, umami | tasteless | — |
| Arg | | | | |
| (SEQ ID NO: 17) | | | | |
| Arg-Ala-Glu-Asp- | 247 | kokumi, umami | 610 | sour, |
| Thr-Ala-Thr-Tyr- | | | | astringent |
| Tyr | | | | |
| (SEQ ID NO: 18) | | | | |
| Glu-Gly-Asp-Ser- | 212 | umami, kokumi | tasteless | — |
| Ala-Ala-Ile | | | | |
| (SEQ ID NO: 19) | | | | |
| Glu-Pro-Glu-Gly- | 61 | umami, kokumi | 243 | astringent, |
| Asp-Val-His-Gln | | | | bitter |
| (SEQ ID NO: 20) | | | | |
| Glu-Asp-Glu-Val- | 96 | umami, sweet, | 894 | sour, |
| Leu-Ala-Thr-Pro | | kokumi | | bitter |
| (SEQ ID NO: 21) | | | | |

From the results, the taste threshold concentrations ranged from 100-1024 μmol/L in water. 7 of 21 peptides did not show any intrinsic taste up to concentrations of 2 mmol/L (i.e., the peptides having SEQ ID NOs: 1, 6, 8, 14, 15, 17, and 19).

In addition, all 21 peptides showed taste modulating properties in the range of 26 μmol/L to 351 μmol/L in model broth. The peptide having SEQ ID NO: 14 has the lowest umami and kokumi threshold of 26 μmol/L. The peptide having SEQ ID NO: 8 has kokumi and salty enhancing taste properties with the highest threshold of 351 μmol/L. The peptide having SEQ ID NOs: 3, 7, and 20 have kokumi, umami and salty modulating taste properties with thresholds of 236, 95 and 61 μmol/L, respectively. The peptide having SEQ ID NOs: 12, 17, 18, 19, and 20 show kokumi and umami enhancing properties with thresholds of 137, 101, 247, 212 and 61 μmol/L.

Besides, the peptide having SEQ ID Nos: 1, 3, 6, 8, 11, 13, 14, 16, and 21, which exhibited the salt-taste modulating properties, were chosen for salt-isointensity tests to evaluate their saltiness enhancing or reducing properties as further described.

### 3. Salt Isointensity Test of Peptides

To investigate the effects of peptides on the perceived saltiness, a salt isointensity test was performed according to literatures. In salt-isointensity test, the peptides (1 mmol/L) were spiked into model broth containing 50 mM NaCl and the panelists were asked to evaluate the intensity of saltiness compared with the following NaCl concentrations of 30, 40, 50, 60, 70, and 80 mmol/L. The results are shown in Table 5.

TABLE 5

| | Average | | |
|---|---|---|---|
| | perceived | | Enhancement |
| | salt | | or |
| | isointensity | RSD | reduction |
| Peptide sequence | (mmol/L NaCl) | (%) | (%) |
| Pro-Val-Leu-Lys (SEQ ID NO: | 42.9 | 4.5 | 14 |
| 1) | | | |
| Glu-Ala-Glu-Phe-Asp (SEQ ID | 43.0 | 6.9 | 14 |
| NO: 3) | | | |
| Glu-Phe-Asp-Glu-Lys-Pro-Ala- | 60.0 | 3.5 | 20 |
| Asp (SEQ ID NO: 6) | | | |
| Glu-Phe-Asp-Ala-Arg-Pro-Thr | 66.7 | 5.8 | 33 |
| (SEQ ID NO: 8) | | | |
| Glu-Phe-Asp-Glu-Lys-Ala-Pro- | 62.1 | 8.0 | 24 |
| Asp (SEQ ID NO: 11) | | | |
| Glu-Asp-Met-Ala-Pro (SEQ ID | 43.8 | 4.1 | 13 |
| NO: 13) | | | |
| Pro-Ile-Asn-Asp-Asn (SEQ ID | 63.6 | 11.3 | 27 |
| NO: 14) | | | |
| Pro-Val-Ala-Glu (SEQ ID NO: | 62.0 | 3.3 | 24 |
| 16) | | | |
| Glu-Asp-Glu-Val-Leu-Ala-Thr- | 59.8 | 3.6 | 20 |
| Pro (SEQ ID NO: 21) | | | |

The perceived salt isointensity of all peptides was in the range of 42.9 to 66.7 mmol/L NaCl in model broth. The peptide having SEQ ID NOs: 6, 8, 11, 13, 14, 16, and 21 exhibited the salt-tase enhancing properties, whereas the peptide having SEQ ID NOs: 1, 3, and 13 exhibited the salt-tase reducing properties. The strongest salt-enhancement effect was observed for the peptide having SEQ ID NO: 8 with 33% salt enhancement (from 50 to 66.7 mmol/L perceived salt isointensity) and the lowest salt-enhancement effect was observed for the peptide having SEQ ID NO: 21 with 20% salt enhancement (from 50 to 59.8 mmo/L perceived salt isointensity). In addition, the salt-reducing effects were observed for the peptide having SEQ ID NOs: 1, 3, and 13 with 14% and 13% respectively.

### 4. Identification of Key Taste-Active and Taste-Modulating Peptides

To identify the peptides which play a key role on taste-active and taste-modulating effects, not only the 21 newly identified peptides, but also literature-known basic tastants (e.g., amino acids, γ-glutamyl peptides, cations, anions, etc.) were quantified in the hydrolyzed chicken plasma sample. Focusing on the 21 newly identified peptides, the taste quality, taste thresholds, dose over threshold (DOT) factors and concentrations of the peptides in the hydrolyzed chicken plasma sample are shown in Table 6. The taste thresholds determined for the newly identified peptides (those are listed in Table 6) were used for the calculation of DoT factors which are defined as the ratio of the concentration of a substance and its corresponding taste threshold concentration. The DoT factors enable a first estimation of the taste contribution as substances with DoT factors≥1 most likely contribute directly to the perceived taste.

TABLE 6

| | Taste | | |
|---|---|---|---|
| | threshold | Peptide | |
| | in model | concentration | |
| | broth | in the sample | DoT |
| Peptide sequence | (μmol/L) | (μmol/kg) | factor |
| Pro-Val-Leu-Lys (SEQ ID NO: 1) | 296 | 2.1 | 0.0 |
| Pro-Leu-Pro-Arg (SEQ ID NO: 2) | 112 | 1.6 | 0.0 |
| Glu-Ala-Glu-Phe-Asp (SEQ ID NO: 3) | 95 | 123.8 | 1.3 |
| Pro-Glu-Arg-Asn-Glu (SEQ ID NO: 4) | 231 | 18.6 | 0.1 |
| Pro-Gln-Pro-Glu-Arg (SEQ ID NO: 5) | 133 | 0.2 | 0.0 |
| Glu-Phe-Asp-Glu-Lys-Pro-Ala-Asp | 94 | 194.1 | 2.1 |
| (SEQ ID NO: 6) | | | |
| Glu-Asp-Ala-Pro-Val-Leu-His | 236 | 13.2 | 0.1 |
| (SEQ ID NO: 7) | | | |
| Glu-Phe-Asp-Ala-Arg-Pro-Thr | 351 | 3.6 | 0.0 |
| (SEQ ID NO: 8) | | | |
| Glu-Glu-Thr-Pro-Ser-His-Asp | 36 | 5.3 | 0.1 |
| (SEQ ID NO: 9) | | | |
| Glu-Glu-Val-Glu-Glu-Glu-Glu-Val-Glu | 34 | 3.2 | 0.1 |
| (SEQ ID NO: 10) | | | |
| Glu-Phe-Asp-Glu-Lys-Ala-Pro-Asp | 67 | 221.1 | 3.3 |
| (SEQ ID NO: 11) | | | |
| Glu-Asn-Ala-Pro-Asp-Gln-Lys | 137 | 20.8 | 0.2 |
| (SEQ ID NO: 12) | | | |
| Glu-Asp-Met-Ala-Pro (SEQ ID NO: 13) | 151 | 27.5 | 0.2 |
| Pro-Ile-Asn-Asp-Asn (SEQ ID NO: 14) | 26 | 102.6 | 3.9 |
| Pro-Thr-Asp-Leu (SEQ ID NO: 15) | 97 | 995.6 | 10.3 |
| Pro-Val-Ala-Glu (SEQ ID NO: 16) | 243 | 171.7 | 0.7 |
| Arg-Asn-Gly-Pro-Arg (SEQ ID NO: 17) | 101 | 0.6 | 0.0 |
| Arg-Ala-Glu-Asp-Thr-Ala-Thr-Tyr-Tyr | 247 | 6.7 | 0.0 |
| (SEQ ID NO: 18) | | | |
| Glu-Gly-Asp-Ser-Ala-Ala-Ile | 212 | 47.5 | 0.2 |
| (SEQ ID NO: 19) | | | |

From the results, five peptides with DoT factors≥1 (SEQ ID NOs: 3, 6, 11, 14, and 15) played a key role on taste-active and taste-modulating effects in the plasma sample.

### Example 3: The Taste-Modulating Effects of Identified Peptides in Food Products

To investigate the taste-modulating performance of the identified peptides in food products, the commercial chicken soup and model broth were used for further sensory experiments. According to the sensory experiment, the peptides such as Pro-Val-Leu-Lys (SEQ ID NO: 1), Pro-Leu-Pro-Arg (SEQ ID NO: 2), Glu-Ala-Glu-Phe-Asp (SEQ ID NO: 3), Pro-Gln-Pro-Glu-Arg (SEQ ID NO: 5), and Glu-Asp-Ala-Pro-Val-Leu-His (SEQ ID NO: 7) are spiked into the commercial chicken soup (Netto Marken-Discount AG&CO.KG, Maxhütte-Haidhof) and the model broth. According to the experiments, 3 g of the commercial chicken soup powder was dissolved in 150 mL boiling water. The peptides (each 2 mmol/L) were spiked into 20 mL solution of this solution. Afterwards, the taste threshold was determined by means of duo-trio-tests as previously described (using chicken soup as reference). Table 7 shows the sensory evaluation of identified peptides in the commercial chicken product and model broth.

TABLE 7

| | Taste | |
|---|---|---|
| | threshold in | |
| | food products | |
| | (μmol/L) | |
| | Commercial | |
| | chicken | Model |
| Peptide sequence | soup | broth |
| Pro-Val-Leu-Lys (SEQ ID NO: 1) | 1500 | 296 |
| Pro-Leu-Pro-Arg (SEQ ID NO: 2) | 418 | 112 |
| Glu-Ala-Glu-Phe-Asp (SEQ ID NO: 3) | 1000 | 95 |
| Pro-Gln-Pro-Glu-Arg (SEQ ID NO: 5) | No effect | 133 |
| Glu-Asp-Ala-Pro-Val-Leu-His (SEQ | 1500 | 236 |
| ID NO: 7) | | |

From the results, the peptide having SEQ ID NOs: 1, 3, and 7 could affect the taste profile of commercial chicken soup at around 1500 μmol/L. In case of the peptide having SEQ ID NO: 5, the spiked sample could not be differentiated (the max concentration tested is 2 mmol/L). The peptide having SEQ ID NO: 2 could be perceived at the concentration of 418 μmol/L in commercial chicken soup, whereas in model broth the threshold was 4 times lower (112 μmol/L). Based on these experimental results, it can be concluded that the peptide having specific amino acid sequences according to the present invention can enhance the taste profile of commercial food products although higher concentrations are necessary compared to model broth.

In addition, further commercial food products with different matrix such as chicken broth (Netto Marken-Discount AG&CO. KG, Maxhütte-Haidhof), vegetable broth (Hügli Nahrungsmittel GmbH, Radolfzell), and pepper sauce (Unilever Deutschland, Hamburg) purchased from local supermarket were used to evaluate the taste-modulating effects of the novel identified peptides according to the present invention, for example, Glu-Ala-Glu-Phe-Asp (SEQ ID NO: 3), Glu-Phe-Asp-Glu-Lys-Pro-Ala-Asp (SEQ ID NO: 6), Glu-Phe-Asp-Glu-Lys-Ala-Pro-Asp (SEQ ID NO: 11), Pro-Ile-Asn-Asp-Asn (SEQ ID NO: 14), and Pro-Thr-Asp-Leu (SEQ ID NO: 15). The food samples were prepared according to the detailed instructions listed on the different products. For chicken broth, and vegetable broth, 3 g of the sample were dissolved in 150 mL boiling water and cooled in an ice bath to room temperature. For pepper sauce, 4 g of the sample were dissolved in 150 mL boiling water. Then, 200 μmol/L of the peptides were spiked into 20 mL broth or sauce. 3-AFC-tests were designed to test the taste properties of the peptides in each commercial product: two samples contained only 20 mL of the commercial product and one sample was 20 mL commercial product spiked with the peptides. The panel was asked to choose the differing sample. Table 8 shows the results of the sensory tests for three commercial food products spiked with the exemplary five peptides.

TABLE 8

| | Number of panelists detecting | Is there a |
|---|---|---|
| | a taste difference from the | significant |
| Food sample | total number of 9 panelists | difference? |
| chicken broth | 7 | Yes |
| vegetable broth | 7 | Yes |
| peper sauce | 8 | Yes |

The results clearly show that the novel identified peptides according to the present invention can be used as the taste-modulating agents in edible products, particularly food or even pharmaceutical products.

### Example 4: Effect of Enzyme Type on Peptide Sequences

To investigate the effect of enzyme types on the amino sequence of peptides presented in the hydrolyzed plasma sample, the chicken plasma was digested with different enzymes such as flavourzyme, and thermolysin.

For flavorzyme digestion, 0.0504 g chicken plasma was extracted with methanol for three times and the sample was dried under nitrogen. The sample was redissolved in 1 M TEAB and urea was added to a final concentration of 8 M. Next, 100 mM DTT was added and kept at 56° C. for 45 min, followed by addition of 0.55 M IAA and incubation at room temperature in the dark for 1 h. Then, the sample was digested with flavorzyme for 15 or 30 min at 37° C. The digestion was stopped by adding concentrated formic acid and the peptide mixture was purified by means of SPE on C18 cartridges.

For thermolysin digestion, plasma sample was prior prepared by adjusting pH to 8, then incubated at 55° C. with thermolysin for 18 h. The reaction was stopped by heating in water bath at 100° C. for 15 min. The solutions were further dried before analysis

The peptide identification was performed by de novo sequencing of LC-TOF-MS data using PEAKS as previously described for screening the 21 peptides (SEQ ID NOs: 1-21, Table 3) in the hydrolyzed chicken plasma samples. Due to flavorzyme's high activity, the chicken plasma was digested with flavorzyme either for 15 or 30 min.

Table 9 shows the exemplary peptides identified in chicken plasma after flavorzyme digestion, and Table 10 shows the exemplary peptides identified in chicken plasma after thermolysin digestion.

TABLE 9

| Digestion | | Mass | RT |
|---|---|---|---|
| time (min) | Peptide sequence | (m/z) | (min) |
| 15 | Gly-Gly-His-Thr-Asp-Ser-Val-Glu-Ala-Gly-Pro- | 2168.0 | 29.0 |
| | Gly-Ile-Asn-Asp-Asp-Gly-Ser-Gly-Ile-Ile-Ser- | | |
| | Asn | | |
| | Asp-Ser-Gly-Val-Asn-Val-Asp-His-Glu-Glu-Pro- | 1588.7 | 22.7 |
| | His-Glu-Glu-Gly-Arg | | |
| | Gly-Ile-Asn-Asp-Pro-Thr-Glu-Gly-Pro-Arg-Thr- | 2300.1 | 36.3 |
| | Val-Ile-Ser-Asp-Pro-Trp-Asp-Ser-Ser-Ala-Ser | | |
| | Glu-Val-His-Ala-Ile-Gly-Ala-Val-Trp-Ala-Asn- | 3101.6 | 43.9 |
| | Val-Leu-Tyr-Glu-Leu-Leu-Trp-Asn-Leu-Ile-Asp- | | |
| | Lys-His-Gly-Lys-Asn | | |
| | Gly-Ile-Asn-Asp-Pro-Thr-Glu-Gly-Pro-Arg-Thr- | 2371.1 | 36.8 |
| | Val-Ile-Ser-Asp-Pro-Trp-Asp-Ser-Ser-Ala-Ser- | | |
| | Ala | | |
| | Asp-Ser-Gly-Val-Asn-Val-Asp-His-Glu-Glu-Pro- | 1746.7 | 22.9 |
| | His-Glu-Glu-Gly-Arg-Ala-Ser | | |
| | Val-Asp-Ser-Gly-Val-Asn-Val-Asp-His-Glu-Glu- | 1845.8 | 23.9 |
| | Pro-His-Glu-Glu-Gly-Arg-Ala-Ser | | |
| | Val-Asp-Ser-Gly-Val-Asn-Val-Asp-His-Glu-Glu- | 1687.7 | 23.8 |
| | Pro-His-Glu-Glu-Gly-Arg | | |
| | Ser-Asp-Leu-Asp-Tyr-Arg-Leu-Pro-Val-Ser-Glu- | 1549.8 | 36.5 |
| | Gly-Leu-Ser | | |
| | Val-Glu-Ala-Gly-Pro-Gly-Ile-Asn-Asp-Asp-Gly- | 1613.8 | 29.7 |
| | Ser-Gly-Ile-Ile-Ser-Asn | | |
| | Phe-Glu-Asp-Tyr-Tyr-Asp-Ser-Ile-Asp-Leu-Pro- | 1823.8 | 42.5 |
| | His-Ile-Pro-Thr | | |
| 30 | Asp-Thr-Val-Asn-Tyr-Leu-Tyr-Glu | 1015.5 | 30.2 |
| | Gly-Ala-His-Gln-Asp-Ser-Ile-Asn-Leu-Asp-Ser- | 2823.3 | 28.5 |
| | Pro-Ser-Glu-Gly-Arg-Ala-Pro-Gly-Ala-Asp-Asp- | | |
| | Asp-Gly-Ser-Gly-Val-Val-Thr | | |
| | Trp-Asp-Thr-Asn-Pro-Pro-Ser-Gly-Pro-Ser-Ala- | 1258.5 | 24.0 |
| | Ser-Pro-Ser | | |
| | Ala-Leu-Val-Ser-Pro-Asp-Glu-Phe-Pro-Glu-Asp | 1217.5 | 33.8 |
| | Asp-Tyr-Arg-Leu-Pro-Val-Ser-Glu-Gly-Leu-Ser | 1234.6 | 33.0 |
| | Ala-His-Gln-Asp-Ser-Ile-Asn-Leu-Asp-Ser-Pro- | 2992.4 | 38.2 |
| | Ser-Glu-Gly-Arg-Ala-Pro-Gly-Ala-Asp-Asp-Asp- | | |
| | Gly-Ser-Gly-Val-Val-Thr-Ile-Leu | | |
| | Phe-Glu-Ser-Ala-Phe-Gly-Asp-Asp-Ser-Pro-Tyr- | 1756.7 | 33.3 |
| | Ile-His-Ser-Ala-Asp | | |
| | Asn-Pro-Pro-Ser-Gly-Pro-Ser-Ala-Ser-Pro-Ser- | 1393.6 | 28.5 |
| | Trp-Thr-Ile-His | | |
| | Tyr-Lys-Gln-Pro-Gln-Val-His | 898.5 | 16.4 |
| | Glu-Phe-Pro-Glu-Asp-Ile-Gln-Leu-Glu | 1118.5 | 37.6 |
| | Val-Pro-Asp-Ser-Ser-Lys-Thr-Tyr | 895.4 | 6.4 |

In case of flavorzyme digestion, it was found that the length of the peptides identified in 15 min digestion was higher compared to the 30 min digestion. With longer time, the digestion is processed more thoroughly, thus producing a higher number of shorter peptides.

TABLE 10

| Peptide sequence | Mass (m/z) | RT (min) |
|---|---|---|
| Leu-Pro-Glu-Asp-Arg-Arg | 784.4191 | 2.08 |
| Leu-Pro-Glu-Asp-Arg | 628.318 | 2.26 |
| Thr-Pro-Asp-Asn-Val-Leu-Leu-Lys | 898.5124 | 23.48 |
| Glu-THR-GLY-LYS-LYS | 561.3122 | 0.84 |
| Val-Gly-Thr-Glu-Glu-Asp-Lys | 776.3552 | 1.46 |

In addition, targeted LC-MS/MS analysis of the 21 peptides, which are the novel identified peptides according to the present invention (SEQ ID NOs: 1-21, Table 3), in the flavorzyme and thermolysin digests revealed that none of these peptides was present due to different enzymes' behavior.

What is claimed is:

1. An isolated peptide having an amino acid sequence selected from the group consisting of:

| (SEQ ID NO: 1) | |
|---|---|
| | Pro-Val-Leu-Lys, |
| (SEQ ID NO: 2) | |
| | Pro-Leu-Pro-Arg, |
| (SEQ ID NO: 3) | |
| | Glu-Ala-Glu-Phe-Asp, |
| (SEQ ID NO: 4) | |
| | Pro-Glu-Arg-Asn-Glu, |
| (SEQ ID NO: 5) | |
| | Pro-Gln-Pro-Glu-Arg, |
| (SEQ ID NO: 6) | |
| | Glu-Phe-Asp-Glu-Lys-Pro-Ala-Asp, |
| (SEQ ID NO: 7) | |
| | Glu-Asp-Ala-Pro-Val-Leu-His, |
| (SEQ ID NO: 8) | |
| | Glu-Phe-Asp-Ala-Arg-Pro-Thr, |
| (SEQ ID NO: 9) | |
| | Glu-Glu-Thr-Pro-Ser-His-Asp, |
| (SEQ ID NO: 10) | |
| | Glu-Glu-Val-Glu-Glu-Glu-Glu-Val-Glu, |
| (SEQ ID NO: 11) | |
| | Glu-Phe-Asp-Glu-Lys-Ala-Pro-Asp, |
| (SEQ ID NO: 12) | |
| | Glu-Asn-Ala-Pro-Asp-Gln-Lys, |
| (SEQ ID NO: 13) | |
| | Glu-Asp-Met-Ala-Pro, |
| (SEQ ID NO: 14) | |
| | Pro-Ile-Asn-Asp-Asn, |
| (SEQ ID NO: 15) | |
| | Pro-Thr-Asp-Leu, |
| (SEQ ID NO: 16) | |
| | Pro-Val-Ala-Glu, |
| (SEQ ID NO: 17) | |
| | Arg-Asn-Gly-Pro-Arg, |
| (SEQ ID NO: 18) | |
| | Arg-Ala-Glu-Asp-Thr-Ala-Thr-Tyr-Tyr, |
| (SEQ ID NO: 19) | |
| | Glu-Gly-Asp-Ser-Ala-Ala-Ile, |
| (SEQ ID NO: 20) | |
| | Glu-Pro-Glu-Gly-Asp-Val-His-Gln, |
| | and |
| (SEQ ID NO: 21) | |
| | Glu-Asp-Glu-Val-Leu-Ala-Thr-Pro. |

2. The isolated peptide of claim 1, which imparts at least one of the following effects salt-reducing, salt-enhancing, umami, and kokumi effects.

3. The isolated peptide of claim 1, which imparts the salt-reducing effect having the amino acid sequence selected from the group consisting of:

| (SEQ ID NO: 1) | |
|---|---|
| | Pro-Val-Leu-Lys, |
| (SEQ ID NO: 3) | |
| | Glu-Ala-Glu-Phe-Asp, |
| | and |
| (SEQ ID NO: 13) | |
| | Glu-Asp-Met-Ala-Pro. |

4. The isolated peptide of claim 1, which imparts the salt-enhancing effect having the amino acid sequence selected from the group consisting of:

| (SEQ ID NO: 6) | |
|---|---|
| | Glu-Phe-Asp-Glu-Lys-Pro-Ala-Asp, |
| (SEQ ID NO: 8) | |
| | Glu-Phe-Asp-Ala-Arg-Pro-Thr, |
| (SEQ ID NO: 11) | |
| | Glu-Phe-Asp-Glu-Lys-Ala-Pro-Asp, |
| (SEQ ID NO: 14) | |
| | Pro-Ile-Asn-Asp-Asn, |
| (SEQ ID NO: 16) | |
| | Pro-Val-Ala-Glu, |
| | and |
| (SEQ ID NO: 21) | |
| | Glu-Asp-Glu-Val-Leu-Ala-Thr-Pro. |

5. The isolated peptide of claim 1, which imparts the umami effect having the amino acid sequence selected from the group consisting of:

| (SEQ ID NO: 1) | |
|---|---|
| | Pro-Val-Leu-Lys, |
| (SEQ ID NO: 2) | |
| | Pro-Leu-Pro-Arg, |
| (SEQ ID NO: 3) | |
| | Glu-Ala-Glu-Phe-Asp, |
| (SEQ ID NO: 4) | |
| | Pro-Glu-Arg-Asn-Glu, |
| (SEQ ID NO: 5) | |
| | Pro-Gln-Pro-Glu-Arg, |
| (SEQ ID NO: 6) | |
| | Glu-Phe-Asp-Glu-Lys-Pro-Ala-Asp, |
| (SEQ ID NO: 7) | |
| | Glu-Asp-Ala-Pro-Val-Leu-His, |
| (SEQ ID NO: 9) | |
| | Glu-Glu-Thr-Pro-Ser-His-Asp, |
| (SEQ ID NO: 10) | |
| | Glu-Glu-Val-Glu-Glu-Glu-Glu-Val-Glu, |
| (SEQ ID NO: 11) | |
| | Glu-Phe-Asp-Glu-Lys-Ala-Pro-Asp, |
| (SEQ ID NO: 12) | |
| | Glu-Asn-Ala-Pro-Asp-Gln-Lys, |
| (SEQ ID NO: 14) | |
| | Pro-Ile-Asn-Asp-Asn, |
| (SEQ ID NO: 17) | |
| | Arg-Asn-Gly-Pro-Arg, |
| (SEQ ID NO: 18) | |
| | Arg-Ala-Glu-Asp-Thr-Ala-Thr-Tyr-Tyr, |
| (SEQ ID NO: 19) | |
| | Glu-Gly-Asp-Ser-Ala-Ala-Ile, |
| (SEQ ID NO: 20) | |
| | Glu-Pro-Glu-Gly-Asp-Val-His-Gln, |
| | and |
| (SEQ ID NO: 21) | |
| | Glu-Asp-Glu-Val-Leu-Ala-Thr-Pro. |

6. The isolated peptide of claim 1, which imparts the kokumi effect having the amino acid sequence selected from the group consisting of:

| (SEQ ID NO: 1) | |
|---|---|
| | Pro-Val-Leu-Lys, |
| (SEQ ID NO: 2) | |
| | Pro-Leu-Pro-Arg, |
| (SEQ ID NO: 3) | |
| | Glu-Ala-Glu-Phe-Asp, |
| (SEQ ID NO: 4) | |
| | Pro-Glu-Arg-Asn-Glu, |
| (SEQ ID NO: 5) | |
| | Pro-Gln-Pro-Glu-Arg, |
| (SEQ ID NO: 7) | |
| | Glu-Asp-Ala-Pro-Val-Leu-His, |
| (SEQ ID NO: 8) | |
| | Glu-Phe-Asp-Ala-Arg-Pro-Thr, |
| (SEQ ID NO: 9) | |
| | Glu-Glu-Thr-Pro-Ser-His-Asp, |
| (SEQ ID NO: 10) | |
| | Glu-Glu-Val-Glu-Glu-Glu-Glu-Val-Glu, |
| (SEQ ID NO: 11) | |
| | Glu-Phe-Asp-Glu-Lys-Ala-Pro-Asp, |
| (SEQ ID NO: 12) | |
| | Glu-Asn-Ala-Pro-Asp-Gln-Lys, |
| (SEQ ID NO: 13) | |
| | Glu-Asp-Met-Ala-Pro, |
| (SEQ ID NO: 15) | |
| | Pro-Thr-Asp-Leu, |
| (SEQ ID NO: 16) | |
| | Pro-Val-Ala-Glu, |
| (SEQ ID NO: 17) | |
| | Arg-Asn-Gly-Pro-Arg, |
| (SEQ ID NO: 18) | |
| | Arg-Ala-Glu-Asp-Thr-Ala-Thr-Tyr-Tyr, |
| (SEQ ID NO: 19) | |
| | Glu-Gly-Asp-Ser-Ala-Ala-Ile, |
| (SEQ ID NO: 20) | |
| | Glu-Pro-Glu-Gly-Asp-Val-His-Gln, |
| | and |
| (SEQ ID NO: 21) | |
| | Glu-Asp-Glu-Val-Leu-Ala-Thr-Pro. |

7. The isolated peptide of claim 1, which is obtained from an enzymatic hydrolysis or a fermentation.

8. The isolated peptide of claim 7, which is obtained from the enzymatic hydrolysis of animal blood.

9. The isolated peptide of claim 8, wherein the animal blood is poultry blood.

10. The isolated peptide of claim 8, wherein the enzymatic hydrolysis is carried out by using serine protease.

11. The isolated peptide of claim 10, wherein the serine protease is subtilisin.

12. Use of the isolated peptide as defined in claim 1 as a taste-modulating agent for a food or pharmaceutical preparation.

13. A protein hydrolysate comprising at least one peptide having an amino acid sequence selected from the group consisting of:

| (SEQ ID NO: 1) | |
|---|---|
| | Pro-Val-Leu-Lys, |
| (SEQ ID NO: 2) | |
| | Pro-Leu-Pro-Arg, |
| (SEQ ID NO: 3) | |
| | Glu-Ala-Glu-Phe-Asp, |
| (SEQ ID NO: 4) | |
| | Pro-Glu-Arg-Asn-Glu, |
| (SEQ ID NO: 5) | |
| | Pro-Gln-Pro-Glu-Arg, |
| (SEQ ID NO: 6) | |
| | Glu-Phe-Asp-Glu-Lys-Pro-Ala-Asp, |
| (SEQ ID NO: 7) | |
| | Glu-Asp-Ala-Pro-Val-Leu-His, |
| (SEQ ID NO: 8) | |
| | Glu-Phe-Asp-Ala-Arg-Pro-Thr, |
| (SEQ ID NO: 9) | |
| | Glu-Glu-Thr-Pro-Ser-His-Asp, |
| (SEQ ID NO: 10) | |
| | Glu-Glu-Val-Glu-Glu-Glu-Glu-Val-Glu, |
| (SEQ ID NO: 11) | |
| | Glu-Phe-Asp-Glu-Lys-Ala-Pro-Asp, |
| (SEQ ID NO: 12) | |
| | Glu-Asn-Ala-Pro-Asp-Gln-Lys, |
| (SEQ ID NO: 13) | |
| | Glu-Asp-Met-Ala-Pro, |
| (SEQ ID NO: 14) | |
| | Pro-Ile-Asn-Asp-Asn, |
| (SEQ ID NO: 15) | |
| | Pro-Thr-Asp-Leu, |
| (SEQ ID NO: 16) | |
| | Pro-Val-Ala-Glu, |
| (SEQ ID NO: 17) | |
| | Arg-Asn-Gly-Pro-Arg, |
| (SEQ ID NO: 18) | |
| | Arg-Ala-Glu-Asp-Thr-Ala-Thr-Tyr-Tyr, |
| (SEQ ID NO: 19) | |
| | Glu-Gly-Asp-Ser-Ala-Ala-Ile, |
| (SEQ ID NO: 20) | |
| | Glu-Pro-Glu-Gly-Asp-Val-His-Gln, |
| (SEQ ID NO: 21) | |
| | Glu-Asp-Glu-Val-Leu-Ala-Thr-Pro, |

and
a combination thereof.

14. The protein hydrolysate of claim 13, comprising at least the peptide having the amino acid sequence of:

| (SEQ ID NO: 3) | |
|---|---|
| | Glu-Ala-Glu-Phe-Asp, |
| (SEQ ID NO: 6) | |
| | Glu-Phe-Asp-Glu-Lys-Pro-Ala-Asp, |
| (SEQ ID NO: 11) | |
| | Glu-Phe-Asp-Glu-Lys-Ala-Pro-Asp, |
| (SEQ ID NO: 14) | |
| | Pro-Ile-Asn-Asp-Asn, |
| | and |
| (SEQ ID NO: 15) | |
| | Pro-Thr-Asp-Leu. |

15. The protein hydrolysate of claim 13, which is obtained by an enzymatic hydrolysis.

16. The protein hydrolysate of claim 15, which is obtained by the enzymatic hydrolysis of animal blood.

17. The protein hydrolysate of claim 16, wherein the animal blood is poultry blood.

18. The protein hydrolysate of claim 16, wherein the enzymatic hydrolysis is carried out by using serine protease.

19. The protein hydrolysate of claim 18, wherein the serine protease is subtilisin.

20. Use of the protein hydrolysate as defined in claim 13 as a taste-modulating agent for a food or pharmaceutical preparation.

21. A taste-modulating composition comprising the isolated peptide as defined in claim 1.

22. A taste-modulating composition comprising the protein hydrolysate as defined in claim 13.

23. Use of the taste-modulating composition of claim 21 in a food or pharmaceutical preparation.

24. A food preparation comprising the isolated peptide as defined in claim 1.

25. A food preparation comprising the protein hydrolysate as defined in claim 13.

26. A food preparation comprising the taste-modulating composition as defined in claim 21.

27. A pharmaceutical preparation comprising the isolated peptide as defined in claim 1.

28. A pharmaceutical preparation comprising the protein hydrolysate as defined in claim 13.

29. A pharmaceutical preparation comprising the taste-modulating composition as defined in claim 21.

30. A method for adjusting a taste of a food preparation, comprising applying an effective amount of the isolated peptide as defined in claim 1 to the food preparation.

31. A method for adjusting a taste of a food preparation, comprising applying the protein hydrolysate as defined in claim 13 which comprises an effective amount of the isolated peptide to the food preparation.

32. A method for adjusting a taste of a food preparation, comprising applying the taste-modulating composition as defined in claim 21 which comprises an effective amount of the isolated peptide to the food preparation.

33. A method for adjusting a taste of a pharmaceutical preparation, comprising applying an effective amount of the isolated peptide as defined in claim 1 to the pharmaceutical preparation.

34. A method for adjusting a taste of a pharmaceutical preparation, comprising applying the protein hydrolysate as defined in claim 13 which comprises an effective amount of the isolated peptide to the pharmaceutical preparation.

35. A method for adjusting a taste of a pharmaceutical preparation, comprising applying the taste-modulating composition as defined in claim 21 which comprises an effective amount of the isolated peptide to the pharmaceutical preparation.
